(19) United States
(12) Patent Application Publication
(10) Pub. No.: US 2022/0193162 A1
(43) Pub. Date: Jun. 23, 2022

(54) FUNGAL EXTRACTS AND FLAVOR COMBINATIONS THEREOF

(71) Applicant: MycoTechnology, Inc., Aurora, CO (US)

(72) Inventors: Anthony J. CLARK, Aurora, CO (US); James Patrick LANGAN, Aurora, CO (US); Ashley HAN, Aurora, CO (US); Lisa SCHMIDT, Aurora, CO (US)

(73) Assignee: MycoTechnology, Inc., Aurora, CO (US)

(21) Appl. No.: 17/600,927

(22) PCT Filed: Apr. 16, 2020
(86) PCT No.: PCT/US2020/028495

§ 371 Date: Oct. 1, 2021

Related U.S. Application Data
(60) Provisional application No. 62/834,858, filed on Apr. 16, 2019.
(60) Provisional application No. 62/836,919, filed on Apr. 22, 2019.

(51) Int. Cl. A61K 36/068; A23L 27/00; A23L 27/10

(52) U.S. Cl. CPC A61K 36/068; A23L 27/84; A23L 27/10; A23L 27/88; A23L 27/86

(57) ABSTRACT

Disclosed is a composition comprising a combination of at least one flavor compound and a taste-modulating portion of a filamentous fungus mycelial aqueous culture, e.g. a mycelia is from a Cordyceps spp, where the flavor compound is a combination and sodium chloride and potassium chloride and when combined with food, beverage or other product, reduces metallic and/or bitter taste resulting from the presence of KCl and provides an extended “salt” flavor perception in the product. The flavor compound can also include at where the at least one flavor compound is a bitterness blocker or a bitterness masker, wherein the combination is capable of synergistically reducing bitter tastes or reducing aftertastes of a food, beverage, or other product. Methods to enhance the taste of a food, beverage or other product using the flavor compounds and taste-modifying portions of a filamentous fungus are also disclosed.

### CROSS RELATED APPLICATIONS

This application claims priority to and the benefit of U.S. Provisional Application Ser. No. 62/834,858, filed Apr. 16, 2019, and 62/836,919 filed Apr. 22, 2019, which are incorporated herein by reference in its entireties.

### BACKGROUND OF THE INVENTION

Human beings have five basic taste modalities: sweet, salty, sour, umami (savory), and bitter. It is known that bitter taste is mediated by the T2R (also known as the TAS2R) family that belongs to the family of GPCRs. In humans, there are about 25 members of the T2R family that may function as bitter taste receptors. In humans, the 25 bitter taste receptors (T2Rs) are activated by hundreds of structurally diverse bitter compounds, while receptor ligands for four of the five of the 25 T2R members (T2R41, T2R42, T2R45, T2R48, T2R60) are at present unknown. In order to best address bitter flavors, it would be best to have a flavor composition that worked at multiple bitter receptors.

Saltiness is perceived upon tasting sodium and/or potassium chloride (NaCl/KCl). Not only does sodium impart desirable flavor to foods, but it serves many functions in food processing. For these reasons salt is one of the most challenging ingredients to replace from a functional and cost-effective standpoint. Potassium chloride is a logical replacement for salt as it mimics its salty flavor as well as most of salt's functional properties. However, potassium chloride can impart a strong metallic and/or bitter aftertaste which is difficult to mask. Due to its metallic bitterness, the amount of potassium chloride in a composition to replace salt is generally fairly limited.

What is desired are improved compositions and methods to improve the flavor of a product for oral administration, such as a composition that inhibits bitter at multiple bitter receptors and a composition that allows for substitution of at least a portion of sodium chloride with potassium chloride while allowing for salty tastes with a minimum of bitterness. The present invention is directed toward overcoming one or more of the problems discussed above.

### SUMMARY

Disclosed is a composition comprising a combination of at least one flavor compound and a taste-modulating portion of a filamentous fungus mycelial aqueous culture, e.g. a mycelia is from a Cordyceps spp, where the flavor compound is a combination and sodium chloride and potassium chloride and when combined with food, beverage or other product, reduces metallic and/or bitter taste resulting from the presence of KCl and provides an extended “salt” flavor perception in the product. The flavor compound can also include at where the at least one flavor compound is a bitterness blocker or a bitterness masker, wherein the combination is capable of synergistically reducing bitter tastes or reducing aftertastes of a food, beverage, or other product.

Methods to enhance the taste of a food, beverage or other product using the flavor compounds and taste-modifying portions of a filamentous fungus are also disclosed, which include the steps of culturing a mycelial aqueous culture in a media; separating the extracellular material fluid from the mycelial cells; and collecting the extracellular material fluid of the mycelial liquid culture as the mycelia-free taste-modulating portion. The method also includes adding at least one flavor compound to the collected mycelia-free taste-modulating portion or collected extract taste-modulating portion to form a combination; and adding the combination to the product for oral administration in an amount sufficient to enhance the product's taste, wherein the enhancement in taste comprises reducing bitter aftertastes, reducing undesirable aftertastes, and/or reducing astringency; or enhancing the salt linger of the food product while reducing bitter flavors.

### BRIEF DESCRIPTION OF THE DRAWINGS

FIG. 1 shows an appetitive rat-lick study of CLEARTASTE in saccharin at various concentrations.

FIG. 2 shows an aversive rat lick study of CLEARTASTE in citric acid at various concentrations.

FIG. 3 shows an aversive rat lick study of CLEARTASTE in quinine at various concentrations.

FIG. 4 shows CLEARTASTE's effect on R-46 at both 25 and 50 ppm using hydrocortisone.

FIG. 5 shows CLEARTASTE's effect on R-16 bitter taste receptor using amygdalin.

FIG. 6 shows CLEARTASTE's effect on R-10 bitter taste receptor using erythromycin.

FIG. 7 shows CLEARTASTE's effect on R-14 bitter taste receptor using noscapine.

FIG. 8 shows CLEARTASTE's effect on R-38 bitter taste receptor using ethylpyrazine.

FIG. 9 shows CLEARTASTE's effect on R-39 bitter taste receptor using acetaminophen.

FIG. 10 shows CLEARTASTE's effect on R-1, -4, -16, -38, -39, -49 bitter taste receptors using diphenidol.

FIG. 11 shows CLEARTASTE's effect using ethanol.

FIG. 12 shows a T-CATA graph of KCl/Salt flour up to 60 seconds.

FIG. 13 shows a T-CATA graph of KCl/Salt flour treated with 10 ppm CLEARTASTE up to 60 seconds.

FIG. 14 shows a T-CATA graph of KCl/Salt granular mixture up to 60 seconds.

FIG. 15 shows a T-CATA graph of KCl/Salt granular mixture treated with 10 ppm CLEARTASTE up to 60 seconds.

### DETAILED DESCRIPTION OF THE INVENTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

In one embodiment, the present invention is based on the discovery that at least a taste-modulating portion of cultured filamentous fungi, including e.g., an extracellular taste-modulating portion, extract of, or other taste-modulating portion thereof of a filamentous fungal mycelial liquid (e.g., aqueous) culture, from a filamentous fungi including Cordyceps sinensis, can be used as a taste modulator. In some embodiments, the taste-modulating portion is pasteurized or sterilized prior to consumption. Prior to use, the taste-modulating portion can be dried, diluted, concentrated, or used neat in the forms of a concentrate, dried powder, and the like. See, e.g., U.S. Pat. Nos. 9,572,363, 9,572,364, and pending patent application Ser. No. 15/438,576. The taste-modulating portion is also referred to herein to by a number of terms, including “bitter blocker (of the invention),” CLEARTASTE, taste-modulating portion, mycelial culture, cultured material, taste modulator, and other such terms. In some embodiments, the material is referred to as a taste-modifying portion of a filamentous fungal mycelial liquid (e.g., aqueous) culture, or “taste-modifying portion.” Methods to manufacture and produce such taste-modifying taste-modulating portions are disclosed herein.

The present inventors have found that a taste-modifying portion of a mycelial aqueous culture of the present invention is a true bitter “blocker” and appears to “block” perception of elicited bitter tastes at a number of TAS-R2 receptors, such as, for example, at hTAS2R1, hTAS2R3, hTAS2R4, hTAS2R5, hTAS2R7, hTAS2R8, hTAS2R13, hTAS2R14, hTAS2R16, hTAS2R38, hTAS2R40, hTAS2R41, hTAS2R43, hTAS2R44, hTAS2R46, hTAS2R47, and hTAS2R49. The taste-modulating portion may be less active at hTAS2R10 and hTAS2R39. Accordingly, in some embodiments of the invention, one or more additional bitter blockers or bitter taste maskers active at hTAS2R10 and hTAS2R39 may be added to the taste-modifying portion to synergistically increase the bitter blocking capacity of the taste-modifying portion.

Knowledge of the activities of the taste-modulating portion as described herein can be used to identify compositions with improved taste-modifying properties. For example, knowledge of the particular taste receptors at which the taste-modifying portions are capable of blocking bitter perception, are not capable of blocking bitter reception, and/or agonize (e.g., activate, or augment) bitter reception, can lead to the design of compositions with improved properties for blocking and/or masking bitter tastes in products for oral administration (e.g., foods). Such compositions can comprise, e.g., combinations of the taste-modulating portion of a mycelial aqueous culture with a flavor compound (also called flavor material herein).

Therefore, in one embodiment, the present invention is directed to compositions comprising, and methods for making, combinations of at least one taste-modulating portion of a filamentous fungal mycelial liquid (e.g., aqueous) culture, together with at least one flavor compound or material.

In another embodiment, the present invention is directed to compositions comprising, and methods for making, combinations of sodium chloride, potassium chloride, and a taste-modifying portion from a mycelial aqueous culture. In an embodiment, the combination may further comprise at least one flavor compound or material. When the taste-modifying portion culture as described herein is combined with sodium chloride and potassium chloride to form the inventive combinations, using effective amounts of each component, the composition, when combined with a consumable, is capable of reducing and/or reduces metallic and/or bitter taste resulting from the presence of KCl and is capable of providing and/or provides an extended “salt” flavor perception in the consumable, as compared to a combination that does not contain a taste-modifying portion from a mycelial aqueous culture.

Optionally, the combination may further include a flavor compound which is a bitterness blocker or bitterness masker, and the flavor compound, in some embodiments, is capable of, in a supplementary, additive, or synergistic manner, modifying undesirable tastes (e.g., improving undesirable tastes) in products for oral administration. Such undesirable tastes include, such as, for example, bitter tastes, astringent tastes, and/or undesirable aftertastes. The combination is also capable of eliminating metallic tastes in products such as potassium chloride. Reducing these tastes may also be referred to as mitigating taste defects.

A surprising aspect of the invention is the appearance of an extended salty perception upon use of taste modifying portion of the mycelial liquid culture, which can be demonstrated by a T-CATA (temporal check-all-that-apply) graph over 60 seconds. While the bitter perception of a mixture using is reduced over control, a surprising aspect is the extension of a salty taste. For example, as can be seen in FIGS. 11 through 14, the salty taste of an aqueous solution containing KCl and NaCl at about 1% is extended (lasts longer) using the taste modifying portion. For example, between about 40 seconds and 60 seconds, the salt perception has noticeably decreased for the controls. Although salt perception also decreases when the taste modifying portion is used, the dominance of a salty taste (shown in dominance (%)) is noticeably greater than the control after this period of time.

Accordingly, the present invention includes a flavor composition comprising a combination of sodium chloride, potassium chloride, and a taste-modifying portion from a mycelial aqueous culture, wherein the mycelia is, in one embodiment, Cordyceps sinensis, as well as methods to improve a consumable's taste by adding such a combination to the consumable. The composition is capable of improving a taste of the consumable. In embodiments, the combination is capable of providing greater improvements to a taste (such as extended salt flavor perception) of the consumable compared with appropriate controls, such as a control comprising a combination of sodium chloride and potassium chloride alone.

The compositions comprising the combinations may further comprise at least one flavor compound. The flavor compound can be any material used for flavoring known in the art. In an embodiment, the flavor compound includes masking flavors and blocking flavors, and can include bitterness masking flavors and bitterness blocking flavors.

The amount of sodium chloride, potassium chloride, and taste-modulating portion and optional flavor compound used in the combinations of the invention, and the amount of the combination with consumables, will depend on the consumable, the flavor compound, and the effect desired. This means that the amount of sodium chloride, potassium chloride, taste-modulating portion, optional flavor compound, in the combination(s) and the ratios of the same used in a consumable, may vary between very wide limits. The skilled person, based on the teachings in the instant specification, can easily determine appropriate amounts of each component to form an effective combination and the amount of combination to use in a consumable, using only routine experimentation and the ordinary skill of the art.

However, as a general, non-limiting guideline, in the combination itself, the goal is to reduce the amount of sodium chloride, and increase the amount of potassium chloride, to provide for ratios of sodium chloride to potassium chloride of about 1%/99% w/w to about 95%/5% w/w. In some embodiments, the ratio of sodium chloride, relative to potassium chloride, can be between 1% and 95% (with a corresponding amount of potassium chloride), by w/w. Alternatively, the amount of each of sodium chloride and potassium chloride may be determined by final amount in the consumable. For example, in broth, the amount of sodium chloride in the broth and potassium chloride in the broth may be about 0.1%, 0.20%, 0.30%, 0.40%, 0.50%, 0.60%, 0.70%, 0.80%, or, 0.9%, in the consumable, by weight percent or weight volume, depending on whether the consumable is a liquid or a solid product. The corresponding amount of potassium chloride in the broth may be 0.1%, 0.20%, 0.30%, 0.40%, 0.50%, 0.60%, 0.70%, 0.80%, or 0.9% The total amount of the combination of both sodium chloride and potassium chloride in the consumable can be about 0.1%, 0.20%, 0.30%, 0.40%, 0.5%, 0.6%, 0.7%, 0.8%, 0.9%, 1%, 1.2%, 1.4%, 1.6%, 1.8%, 2.0%, 2.5%, 3%, 3.5%, 4%; the proper amount to add will depend on the ordinary level of saltiness or salty flavor in the consumable and can be determined by one of skill in the art. For example, for broth, to replace a level of sodium chloride of about 0.9%, in some embodiments, the amount of sodium chloride is reduced by half in the broth and is approximately 0.45% w/v and the corresponding amount of potassium chloride is about 0.52%. In another embodiment, the amount of sodium chloride in the consumable, relative to an ordinary or customary level of sodium chloride, may be reduced to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% if the usual level.

In embodiments, a formulation of the inventive combination can include a concentrated formula. In the concentrated formula, the concentrations disclosed above for the sodium chloride, the potassium chloride, and the taste-modifying portion can be proportionately scaled to form a concentrated formula. For example, for a broth product, where the concentration of each component in the inventive combination is 0.52% potassium chloride, 0.45% sodium chloride, and 20 ppm taste-modifying portion, the formulation may comprise 52% potassium chloride, 45% sodium chloride, and 200 ppm taste modifying portion, with the remainder of the material an inert ingredient. Formulations with appropriate proportional dilutions of each component in the combination may also be developed, with the remainder of the formulation comprising inert and/or carrier ingredients, such as, for example, a bulking agent, an anti-caking agent, which can include a starch, such as maltodextrin, gum acacia and the like.

In one embodiment, when the taste-modifying portion culture as described herein is combined with a flavor compound or material, such as, for example, a flavor compound which is a bitterness blocker or bitterness masker, the combination is capable of, in a supplementary, additive, or synergistic manner, modifying undesirable tastes (e.g., improving undesirable tastes) in products for oral administration. Such undesirable tastes include, such as, for example, bitter tastes, astringent tastes, and/or undesirable aftertastes. Improving the taste of a product for oral administration includes improved sweetening by a sweetening flavor product, such as a non-nutritive sweetener. Flavor improvement also includes reduction of characteristic aftertastes associated with stevia and tea, including, without limitation, a soapy flavor, a bitter flavor, a metallic flavor, a licorice flavor, commonly as an aftertaste, which sets on after the initial sweet or tea sensation. The combination is also capable of eliminating metallic tastes in products such as potassium chloride. The combination can also be used to reduce undesirable flavor defects in breads and formulations made from various grains such as quinoa, amaranth and whole wheat. Reducing these tastes may also be referred to as mitigating taste defects. For example, steviol glycosides possess residual bitterness and aftertaste, which affect its qualitative characteristics.

Improved flavor of products for oral administration treated by the compositions of the invention may be measured in a variety of ways, such as the chemical analysis which demonstrate improved sweetness, reduced bitterness, reduced astringency, and/or mitigated other taste defects. Taste tests with taste panels may also be conducted to provide qualitative data with respect to improved taste(s) in the compositions in combination with products for oral administration, with the panels determining whether improved sweetness and/or decreased taste defects have been exhibited in the treated products. In an embodiment, the compositions comprising the inventive combinations with products for oral administration have reduced bitterness and/or reduced astringency and/or reduced other undesirable tastes compared to the product for oral administration alone. In an embodiment, the product for oral administration when combined with a composition of the invention has the changed organoleptic perception as disclosed in the present invention, as determined by human sensory testing. It is to be understood that the methods of the invention only optionally include a step of determining whether the flavor of the compositions in combination with products for oral administration differ from a control material. The key determinant is, if measured by methods as disclosed herein, that the compositions in combination with products for oral administration are capable of providing the named differences from control materials.

Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine the organoleptic qualities, e.g., flavor, of the instant invention can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone. In the instant invention, it should be understood that there are any number of ways one of skill in the art could measure the sensory differences.

In an embodiment, the inventive combinations, when used with products for oral administration, provide the products with reduced undesirable tastes, such as reduced bitterness, as measured by sensory testing as known in the art. Such methods include change in taste threshold, change in bitterness intensity, and the like. At least 10% or more of a change (e.g., reduction in) bitterness is preferred. The increase in desirable flavors and/or tastes may be rated as an increase of 1 or more out of a scale of 5 (1 being no taste, 5 being a very strong taste.) Or, a reference may be defined as 5 on a 9 point scale, with reduced bitterness or at least one flavor as 1-4 and increased bitterness or at least one flavor as 6-9.

Accordingly, the present invention includes a composition comprising a combination of a taste-modulating portion of a mycelial aqueous culture with at least one flavor compound, as well as methods to improve a product for oral administration's taste by adding such a combination to a food product. The composition is capable of improving a taste of the product for oral administration. In embodiments, the combination is capable of providing greater improvements to a taste of the product for oral administration compared with the taste-modulating portion of mycelial aqueous culture alone and/or the at least one flavor compound alone. In embodiments, the taste-modulating portion is also capable of improving the taste of a product for oral administration alone.

The compositions comprising the combinations may further comprise a product for oral administration.

In embodiments, the compositions of the invention include at least one flavor compound. The flavor compound can be any material used for flavoring known in the art. In an embodiment, the flavor compound includes masking flavors and blocking flavors, and can include bitterness masking flavors and bitterness blocking flavors.

“Blocking”, as used herein, relates to changing taste attributes by a chemical process. An ingredient that acts as a blocker will interact in one of two ways: 1) it “binds” (for the lack of a better term) with the bitter taste receptor on the tongue or 2) it binds with the “offending” tastant. Wherever this interaction occurs, the “villain” tastant won't be perceived by the tongue—the bitter taste or off-note is blocked. T2Rs are activated by structurally diverse natural and synthetic bitter compounds.

As discussed herein, the present inventors have found that while the taste-modulating portion of mycelial aqueous culture described herein is effective as a bitter blocker, and appears to be a wide spectrum bitter blocker, there are bitter taste receptors that are not blocked by the taste-modulating portion of the invention. In summary, as discussed above, the inventors have found that the taste-modulating portion of mycelial aqueous culture of the invention blocks bitterness due to TAS2R-46 (hydrocortisone) and TAS2R-16 (amygdalin), as well as at TAS2R-14 and TAS2R-38. Further, the inventors also found that the taste-modulating portion as described herein is also capable of reducing bitter taste from activation due to the broad-spectrum bitter agent diphenidol (TAS2R-1, -4, -16, -38, -39, -47, -48). Additionally, the inventors found that the taste-modulating portion as described herein is capable of reducing bitter taste reception due to saccharin, citric acid, and quinine, indicating activity at TASR2-4, -7, -8, -43, -44, and ethanol, indicating activity at TAS2R-3, -4, -5. However, the taste-modulating portion of mycelial aqueous culture of the invention has a lesser effect on bitterness due to TAS2R-10 (erythromycin) and TAS2R-39.

One aspect of the invention therefore provides the use of a combination that can modulate multiple bitter taste receptors (T2R), in combination with, a product for oral administration, e.g., a food, drink, confectionary, oral care, or oral pharmaceutical product, to enhance the taste of the product. Furthermore, the invention contemplates a method of using a combination of the invention in combination with, a product for oral administration. A combination according to the present invention can be used as a pretreatment before the ingestion of a bitter substance or administered contemporaneously with a bitter substance.

Accordingly, according to the invention, the effectiveness of the taste-modulating portion as described herein can be enhanced by combining it with any flavor compound known in the art, such as a bitter blocker and/or a bitter masker to form the inventive combination. In an embodiment, the flavor compound to add to the combination can be selected to additively or synergistically augment the bitter blocking capacity of the taste-modulating portion of mycelial aqueous culture. In one embodiment, a flavor compound that blocks bitterness due to activation of the any of the receptors, including, for example, TAS2R-1, TAS2R-4, TAS2R-5, TAS2R-7, TAS2R-8, TAS2R-10, TAS2R-13, TAS2R-14 TAS2R-16, TAS2R-38, TAS2R-39, TAS2R-40, TAS2R-41, TAS2R-43, TAS2R-44, TAS2R-47, and/or TAS2R-49, or, alternatively, TAS2R-10, TAS2R-39, TAS2R-14, and/or TAS2R-38 receptors can be used in the combination to increase the spectrum of bitterness blocking for the taste-modulating portion. In one embodiment, a flavor compound that blocks bitterness due to activation of TAS2R-10 and/or TAS2R-39 can be used in the combination. In another embodiment, a flavor compound that blocks bitterness due to activation of TAS2R-14 and/or TAS2R-38 can be used in the combination. In some embodiments, the combination's ability to block bitter tastes is synergized due to the addition of a flavor compound that blocks one or more of TAS2R-10, TAS2R-14, TAS2R-38, and TAS2R-39, or one or more of TAS2R-14 and TAS2R-38.

In an embodiment, one or more flavor compounds that are bitter masking agents can be used with the taste-modulating portion of mycelial aqueous culture, and the bitter masking agent can be optionally used together with the combination of the taste-modulating portion of mycelial aqueous culture and bitter blocking agent.

In an embodiment, the bitter masking agent can be any bitter masking agent known in the art, and can optionally include such known bitter masking agents e.g., an agent selected from the group consisting of: sucralose, zinc gluconate, ethyl maltol, glycine, acesulfame K, aspartame, saccharin, fructose, xylitol, malitol, isomalt, salt, spray dried licorice root, glycyrrhizin, dextrose, sodium gluconate, sucrose, glucono-delta-lactone, ethyl vanillin, and/or vanillin, among others.

Alternatively, the bitter taste masker can include one or more any bitter taste masker known in the art, optionally, one known to be active at bitterness due to TAS2R-10 (erythromycin) and TAS2R-39. Maskers include damascenone; 4-hydroxybenzyl alcohol; tocopherol; d-camphor; L-lysine, DL-alanine; L-phenylalanine; phytyl acetate; phytol; 2-methyl-3-(3,4-methylenedioxyphenyl)-propanol; lactic acid; vanillyl alcohol, among others.

A product for oral administration according to the present invention can include a food, drink, confectionary, oral care, or oral pharmaceutical product, and includes food products, food additives, food ingredients, non-caloric sweeteners, salt substitutes, dietary supplements, food additives, pharmaceuticals, foodstuffs, cosmetic ingredients, nutraceutical ingredients, dietary ingredients, and processing aids. Any product for oral administration which has or can have undesirable taste characteristics, such as bitter tastes, undesirable aftertastes, astringent tastes, and the like, can be treated with the combinations of the present invention.

In some embodiments, the food product includes tea plant parts, tea decoctions, or tea purified extracts. Food products that may be included in compositions of the invention include food products according to the invention, and include, for example, non-nutritive sweeteners and nutritive sweeteners. These include, without limitation, non-nutritive sweeteners such as mogroside, mogroside mixtures, aspartame, acesulfame-k, sucralose, steviol glycoside mixtures, stevia plant parts, stevia rebaudioside A, steviol glycoside, stevia plant parts. Sweeteners include non-nutritive and/or artificial sweetening agents, e.g., selected from steviol glycosides, sucralose, neotame; sugar alcohols, e.g. sorbitol and/or xylitol; an artificial sweetener having a bitter aftertaste, e.g., a sulfonyl amide sweetener, e.g., selected from saccharin, sodium cyclamate and acesulfame potassium. Non-limiting examples of natural sweeteners include stevioside (disclosed previously), Luo Han Guo extract, monk fruit, glycyrrhizin, perillartine, naringin dihydrochalcone, neohesperidine dihydrochalcone, rubusoside, rubus extract, and rebaudioside A.

Another category of food products includes, for example, whole wheat, coffee, tea, amaranth, quinoa, pea protein, monk fruit, monk fruit extract, beer, liquor, spirits, wine, sucralose, carbohydrates, potassium chloride, cacao, cacao liquor, ginseng, cranberry, grapefruit, pomegranate, and coconut. Food products may also include coffee, roasted coffee beans, roasted coffee grinds, tea leaves, or brewed tea. Also, food products include protein concentrates, e.g., a product comprising greater than 50% protein. Such a food product can be obtained from a number of sources, including vegetarian sources as well as non-vegetarian sources. Vegetarian sources include protein concentrates and isolates prepared from a vegetarian source such as pea, rice, soy, hemp, and other sources, or a combination thereof. Typically a protein concentrate is made by removing the oil and most of the soluble sugars from a meal made of the starting material, such as soybean meal. A protein concentrate may still contain a significant taste-modulating portion of non-protein material, such as fiber. Typically, protein concentrations in a concentrate are between 65-90%. A protein isolate typically removes most of the non-protein material such as fiber and may contain up to about 90% protein. A protein isolate is typically dried and is available in powdered form and may alternatively called “protein powder.”

In particular, the combinations of the present invention are useful, e.g., in methods as hereinbefore described, to offset the bitter taste of common food ingredients such as potassium chloride, ammonium chloride, sodium chloride, magnesium chloride, halide salts, naringin, caffeine, urea, magnesium sulfate, saccharin, acetosulfames, aspirin, potassium benzoate, potassium bicarbonate, potassium carbonate, potassium nitrate, potassium nitrite, potassium sulfate, potassium sulfite, potassium glutamate, unsweetened chocolate, cocoa beans, yogurt, preservatives, flavor enhancers, dietary supplements, supplemental amino acids, as well as potassium-containing or metal-containing substances with undesirable tastes.

Food products include all cereals, grains, all species of wheat, rye, brown rice, white rice, red rice, gold rice, wild rice, rice, barley, triticale, rice, sorghum, oats, millets, quinoa, buckwheat, fonio, amaranth, teff and durum; apples and pears, apricots, cherries, almonds, peaches, strawberries, raisins, manioc, cacao, banana, Rubiaceae sp. (coffee), lemons, oranges and grapefruit; tomatoes, potatoes, peppers, eggplant,

Finished food products comprising the combinations of the invention also include, but are not limited to, cereal products, rice products, tapioca products, sago products, baker's products, biscuit products, pastry products, bread products, confectionery products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yoghurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof.

Products for oral administration further include nutraceuticals and pharmaceuticals. For example, many medicaments, for example, without limitation, pain medications, include caffeine to enhance their effect, and therefore have a bitter taste. Theophylline is a methylxanthine drug used in therapy for respiratory diseases such as COPD or asthma. Nicotine is a bitter methylxanthine drug used in, for example, chewing gum to help to quit smoking.

Beverages comprising the combinations of the invention include any aqueous drink, enhanced/slightly sweetened water drink, carbonated beverage, non-carbonated beverage, soft drink, non-alcoholic drink, alcoholic drink, fruit drink, juice, fruit juice, vegetable juice, coffee, tea, black tea, green tea, oolong tea, herbal tea, cocoa (water-based), cocoa (milk-based), cocoa (soy-based), tea-based drink, coffee-based drink, cocoa-based drink, syrup, frozen fruit, frozen fruit juice, water-based ice, dairy ice, fruit ice, sorbet, and beverages formed from botanical materials (whole or ground) by brewing, soaking or otherwise extracting, and beverages formed by dissolving instant powders or concentrates (coffee beans, ground coffee, instant coffee, cocoa beans, cocoa powder, instant cocoa, tea leaves, instant tea powder), and the above-mentioned concentrates.

Finished food products include but are not limited to cereals, baked food products, biscuits, bread, breakfast cereal, cereal bar, energy bars/nutritional bars, granola, cakes, cookies, crackers, donuts, muffins, pastries, confectioneries, chewing gum, chocolate, fondant, hard candy, marshmallow, pressed tablets, snack foods, and botanical materials (whole or ground), and instant powders for reconstitution as mentioned herein above.

Dairy products, dairy-derived products and dairy-alternative products include but are not limited to milk, fluid milk, cultured milk product, cultured and noncultured dairy-based drinks, cultured milk product cultured with lactobacillus, yoghurt, yoghurt-based beverage, smoothie, lassi, milk shake, acidified milk, acidified milk beverage, butter milk, kefir, milk-based beverage, milk/juice blend, fermented milk beverage, ice cream, dessert, frozen yoghurt, soy milk, rice milk, soy drink, rice milk drink. Milk includes, but is not limited to, whole milk, skim milk, condensed milk, evaporated milk, reduced fat milk, low fat milk, nonfat milk, and milk solids (which may be fat or nonfat).

In a further embodiment, oral care products of the invention are provided that comprise a combination of the invention and an oral care products.

The amount of taste-modulating portion and flavor compound used in the combinations of the invention, and the amount of the combination with products for oral administration, will depend on the product for oral administration, the flavor compound, the taste-modulating portion, and the effect desired. This means that the amount of taste-modulating portion, flavor compound, and combination used in a product for oral administration, may vary between very wide limits. The skilled person can easily determine an appropriate amount of taste-modulating portion, flavor compound in the combination and the amount of combination to use in the food in every case, using only routine experimentation and the ordinary skill of the art.

However, as a general, non-limiting guideline, for non-nutritive sweeteners, the amount of taste-modulating portion to use with can vary between about 0.1 ppb and about 500 ppm. As a general, non-limiting guideline, for dairy and dairy substitutes, beverages, nutritional bars, vitamins, grains, protein concentrates and isolates, dietary supplements, finished foods, pharmaceuticals, the amount of taste-modulating portion to use with can vary between about 0.1 ppb and about 500 ppm. In embodiments, the range is between about 0.1 ppm and 50 ppm. Further guidelines for use of the taste-modifying portion together can be found in the Examples. The amount of a flavor compound to use in a combination together with the taste-modifying portion for non-nutritive sweeteners may be determined by one of skill in the art, starting with the amounts recommended by other sources, such as publications and technical guidance available in the art.

A general range of concentrations of C. sinensis taste modulating portion (bitter blocker) as a dried powder to use with various food products is also shown in the Examples below. It is within the skill in the art to determine optimum ratios of the inventive combination to use with a particular product, based on taste profiles. For example, at too high concentrations of the inventive combination, the flavor enhancing effect will cease to be or the product will introduce flavor defects into the final material. At too low of a concentration of the inventive combination, there will be an insufficient degree of taste improvement. The concentration of the agricultural material, such as a steviol glycoside mixture which is typically used at 35-450 ppm, ultimately determines the ideal bitter blocker concentration. For example, serial dilution/concentration can be used as a tool in determining the upper and lower threshold concentrations use of the extracellular material.

The taste-modulating portion of mycelial aqueous culture, in embodiments, may be prepared by a method which includes the following steps, in no particular order. The method includes culturing a mycelial aqueous culture in a media; separating the extracellular fluid from the mycelial cells; and collecting the extracellular fluid of the mycelial liquid tissue culture as the mycelia-free taste-modulating portion. The method may also include culturing a mycelial aqueous culture in a media; heating the mycelial aqueous culture to between about 60° C. and 80° C. for between about 40 minutes and 80 minutes; separating the extracellular fluid from the mycelial cells; and collecting the extracellular fluid of the mycelial liquid tissue culture as the extract taste-modulating portion.

In brief, in one embodiment, the mycelial liquid culture is carried out in a bioreactor pressure vessel which is ideally constructed with a torispherical dome, cylindrical body, and spherical cap base, jacketed about the body, equipped with a magnetic drive mixer, and ports through curled-in jacket spaces to provide access for equipment comprising DO probes, pH meters, conductivity meters, thermocouples, etc., as is known in the art.

The reactor preferably is outfitted with a means for sterile inoculation. In one embodiment, to inoculate the reactor, a glycerol stock solution of fungi is used to inoculate the reactor. At least one scale-up reactor can be used before approaching tanks with volumes on the order of 1×10⁵. The inventors recommend going from the order of 1×100 L to 1×10² L to 1×10⁴ L to 1×10⁵⁻⁶ L. Richer media can be used for the scale-up reactors and pre-glycerol stock culturing motifs.

In one embodiment, a fungus strain useful for the fungal component of the present invention in one embodiment is C. sinensis strain WC859, commercially available from Pennsylvania State University (The Pennsylvania State University Mushroom Culture Collection, available from the College of Agriculture Sciences, Department of Plant Pathology and Environmental Microbiology, 117 Buckhout Laboratory, The Pennsylvania State University, University Park, Pa., USA 16802). Fungal components useful in the present invention may be prepared by methods described herein. Other methods known in the art may be used.

Alternatively, the fungal aqueous culture can include other species of fungi from genus Cordyceps, Ophiocordyceps, Elaphocordyceps, Metacordyceps, such as, for example, C. militaris. Many other species exist in the genus, however, these species are generally not cultivated commercially. However, it is expected that, for example, C. scarabaeicola, C. takaomontana, Ophiocordyceps dipterigena, Ophiocordyceps amazonica, C. cylindrica, Cordyceps sphecocephala, Metacordyceps martialis, Ophiocordyceps melonlonthae, Ophiocordyceps nutans, Ophiocordyceps curculionium, Ophiocordyceps australis, Ophiocordyceps tiputini, Cordyceps caloceroides, and Cordyceps variabilis will have the same or similar bitter blocking ability as C. sinensis.

In one embodiment, the invention includes a method for preparing a mycelia-free taste-modulating portion of the mycelial liquid culture after culturing. The mycelia-free taste-modulating portion includes mycelial biomolecular extracellular solids, cellular material and residual media of the mycelial liquid culture.

As disclosed hereinabove, to prepare the culture, the prepared media is inoculated into a container of sterilized human grade media in water.

In some embodiments, the fungal aqueous culture is C. sinensis grown in a liquid submerged media consisting of 8 g/L organic potato starch powder and 0.8 g/L organic carrot powder. This minimal medium has been found by the inventors to be an effective media recipe for producing the bitter blocker (taste enhancement food product) as previously described. The resulting extracellular powder may be used as a bitter blocker in product applications as discussed herein.

After a suitable time for culturing, which can be determined by one of skill in the art, the aqueous culture will comprise mycelia and an extracellular material (external to the mycelia). The taste-modulating portions of the mycelial aqueous culture suitable for the invention include all taste-modulating portions of the aqueous culture, including a whole, undivided taste-modulating portion; a taste-modulating portion comprising a separated extracellular material (also called extracellular fluid or mycelia-free taste-modulating portion), a taste-modulating portion comprising a separated mycelial material. The separated extracellular material taste-modulating portion and separated mycelial material may also be obtained from a heat-treated whole aqueous culture. In other words, the taste-modulating portions of the mycelial aqueous culture of the invention may also include a taste-modulating portion of separated extracellular material (soluble taste-modulating portion, also called extract taste-modulating portion) obtained after a heat treatment step of the whole mycelial aqueous culture or a taste-modulating portion of separated cellular material (insoluble material) obtained after a heat treatment step of the whole mycelial aqueous culture.

Any of the taste-modulating portions of the mycelial aqueous culture as described above may be used in the present invention. Culturing can take place, for example, for between about one and about sixty days, between about two and about fifty days, between about three and about forty days, between about four and about thirty days, between about five and about twenty-five days, between about six and about twenty days, between about seven and about fifteen days, between about eight and about twelve days, and between about nine and about ten days. The length of time for culturing can be determined by, for example, economic considerations for number of days in culture and the degree of taste enhancement observed for a particular culture time.

A mycelial aqueous culture may include any liquid culture comprising mycelia, for example, submerged or floating culture. A submerged culture is generally agitated, whereas the floating culture is minimally agitated, which allows the mycelia to grow in a mat-like form. The taste-modulating portions of the culture to use with the present invention includes any and all parts or taste-modulating portions of the culture, including mycelia, culture extracellular or filtrate, or any taste-modulating portions or fractions thereof.

In one embodiment, after the mycelia has been grown to the desired level in the aqueous culture, the mycelia may be heat treated, e.g., treated to reduce or eliminate the viability of live organisms, using methods such as pasteurization or sterilization, by methods known in the art. In one embodiment the material is sterilized under conditions such as approximately 30 to 50 minute exposure to 250° F. saturated steam at 23 psi. Alternatively, the material can be pasteurized by holding the material in a hot water bath at 160 to 170° F. for 20 minutes, twice, cooling it back to room temperature in between runs.

In some embodiments, the culture may be blended or homogenized (mechanically or by other methods), either before or after the optional sterilization or pasteurization step. The homogenized or blended culture may be used whole, or the soluble taste-modulating portion and/or insoluble taste-modulating portion may be used.

The culture is then further optionally separated to remove the soluble (aqueous) taste-modulating portion from the insoluble taste-modulating portion.

In some embodiments, the taste-modulating portion of the culture to use is the taste-modulating portion of the culture which is soluble, which is commonly understood as the “cell culture extracellular” or “cell culture filtrate”, i.e., the fluid taste-modulating portion of the culture which has been separated from the mycelial cells, and contains a relatively smaller or lesser amount of mycelia as opposed to a mycelial cell taste-modulating portion, which is enriched in mycelial cells, but will still contain some fluid taste-modulating portion. Such soluble taste-modulating portion may also contain taste-modulating portions of the mycelia made soluble due to the optional heat treatment step as described above, due to a pasteurization step or a sterilization step.

Thus, it should be understood that this culture extracellular can commonly contain mycelia, even if not visible to the eye or even easily visible under a microscope. This taste-modulating portion of the culture is called herein the “mycelial-free” taste-modulating portion for convenience, however, as stated it should be understood that this taste-modulating portion will commonly contain some minimal amount of mycelia, even if not visible to the eye.

The separated aqueous culture can be separated to remove soluble and insoluble taste-modulating portions (e.g., mycelia) by any method known in the art to separate cell culture extracellular from cellular materials. For example, the culture may be filtered by any means known in the art to obtain the soluble taste-modulating portion, such as, for example, 0.2 μm filters and the like. Alternatively, the soluble taste-modulating portion of the culture may be collected by centrifugation. The collected soluble taste-modulating portion of the mycelial liquid culture may be referred to herein as collected extracellular material, supernatant, extracellular fluid, C. sinensis supernatant and/or extracellular portion, filtrate, product, and similar terms.

This pasteurized or sterilized liquid culture could be used as a novel beverage, or its powder as a novel foodstuff, food ingredient, dietary supplement, dietary ingredient or food additive which can be used from 0.1-40,000 ppm in various product applications.

The filtrate (collected extracellular portion) e.g., mycelia-free taste-modulating portion of a mycelial liquid culture may have its volume or liquid component adjusted as determined by one of skill in the art to produce concentrates, diluates, or dried powders. In one embodiment, the filtrate may be optionally dried by any method known in the art, including the use of open air drying, small batch desiccators, vacuform dryers, fluid beds or spray dryers, or freeze-driers to dry the liquid to a powder. The filtrate is, in one embodiment, dried following sterilization/pasteurization.

The inventive combination may also be rehydrated, filtered and re-dried to increase solubility of the product. The spray dried product has high solubility and optionally is not rehydrated before use, and may be simply mixed in as a powder with a product for oral administration (particularly in non-nutritive sweetener applications). Alternatively, the inventive combination may be combined with a product in liquid form, and optionally the combination and the product for oral administration may be dried together. The inventive combination may also be dried in a fluid bed, or spray dried onto a fluidized product and even agglomerated, such as in the production of a steviol glycoside mixture comprising the product.

The present invention also provides for a method to enhance the taste of a product for oral administration, comprising a step of culturing a mycelial liquid culture in a media, separating the extracellular fluid from the mycelial culture, collecting the mycelia-free taste-modulating portion of the extracellular material as the taste-modifying portion. The step may alternatively comprise culturing a mycelial liquid culture in a media, heating the mycelial liquid culture to between about 60° C. and about 80° for between about 40 minutes and about 80 minutes; separating the liquid (or extracellular, or soluble) and collecting it as the taste-modulating portion of the culture or the extract portion. Another step includes adding a flavor compound to the collected mycelia-free portion or collected extract to form a combination. Another step includes adding the combination to the product for oral administration in an amount sufficient to enhance the product's taste, wherein the enhancement in taste comprises reducing bitter aftertastes, reducing undesirable aftertastes, and/or reducing astringency. Appropriate fungi to use, appropriate media, appropriate flavor compounds, appropriate methods of collecting the taste-modulating portion are disclosed herein. The taste-modulating portion may be optionally concentrated, diluted or dried as disclosed herein, and may be combined with any flavor compound to form a combination for use with any product for oral administration as disclosed herein prior to use. The present invention also includes combination products comprising one or more products for oral administration, one or more flavor compounds, and a taste-modulating portion made from a mycelial liquid culture made by the processes disclosed herein.

The following examples are provided for illustrative purposes only and are not intended to limit the scope of the invention.

### EXAMPLES

### Example 1

A 4 L flask filled with 1.5 L of 8 g/L organic potato starch and 0.8 g/L organic carrot powder in RO water was sterilized and inoculated from a two week old P1 C. sinensis culture. After culturing for 7 days at room temperature at 60 RPM (1″ swing radius), the culture was filtered through three stacked coffee filters, pasteurized for 40 minutes at 165° F. and placed in a small batch desiccator at 140° F. overnight. The following day the dried material was collected and blended with a yield of 4.5 g/L for a total of 6.75 g. 5 g of the harvested material was poured into 1 L of RO water and shaken intermittently for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm. 150 mg of this mixture was added to 500 mL of RO water to create a solution of 300 ppm 97% rebaudioside A to 0.8 ppm C. sinensis extracellular material solids. When taste tested against a control, testers agreed that the aftertaste of the steviol glycoside mixture containing the C. sinensis extracellular material solids had decreased and was undetectable compared to a control 300 ppm 97% rebaudioside A solution.

### Example 2

A 4 L flask filled with 1.5 L of 8 g/L organic potato starch and 0.8 g/L organic carrot powder in RO water was sterilized and inoculated from a two week old P1 C. sinensis culture. After culturing for 4, 7 or 10 days at room temperature at 60 RPM (1″ swing radius), the culture was filtered through a vacuum filter or through cheesecloth, pasteurized for 50 minutes at 160° F. and placed in a small batch desiccator at 130° F. overnight. The following day the dried material was collected and blended with a yield of 4.4 g/L for a total of 6.6 g. 5 g of the harvested material was poured into 1 L of RO water and shaken intermittently for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm. 150 mg of this mixture was added to 500 mL of RO water to create a solution of 300 ppm 97% rebaudioside A to 0.8 ppm C. sinensis extracellular material solids. When taste tested against a control, in all samples the aftertaste of the steviol glycoside mixture containing the C. sinensis extracellular material solids was undetectable compared to a control 300 ppm 97% rebaudioside A solution.

### Example 3

16 different media recipes to determine the effect of media on bitter blocking activity against a sample of 60% rebaudioside A using the method of Example 2, while varying media as shown below. Table 1 below shows what media were tested and the sensory response summaries.

TABLE 1
Effect of Media on Bitter Blocking Activity against 60% rebaudioside A*

| Media Recipe | Result |
|---|---|
| Nutritional Yeast | No stevia aftertaste, though introduced a new |
| | undesirable aftertaste |
| Brown Rice Syrup | No aftertaste, typical up front flavor, no |
| | new flavors introduced |
| Corn & Oat Flours | No aftertaste, very nice up front stevia flavor |
| | no new flavors introduced |
| Potato Starch Powder | No aftertaste, typical up front stevia flavor, no |
| | new flavors introduced |
| Barley Flour | No aftertaste, duller up front stevia flavor, no |
| | new flavors introduced |
| Kelp | No aftertaste, muted up front stevia flavor, no |
| | new flavors introduced |
| Green Tea | No aftertaste, introduces a tea flavor defect |
| | up front |
| Carrot Powder | No aftertaste, nice up front stevia flavor, no |
| | new flavors introduced |
| Brown Rice Flour | No aftertaste, nice up front stevia flavor, no new |
| | flavors introduced |
| Blackstrap Molasses | No aftertaste, mild up front stevia flavor, no |
| | new flavors introduced |
| Sodium Carboxy- | No aftertaste, mild up front stevia flavor, |
| methylcellulose | no new flavors introduced |
| Wheat Flour | No aftertaste, dull up front stevia flavor, |
| | no new flavors introduced |
| Rye Flour | No aftertaste, dull up front stevia flavor, |
| | no new flavors introduced |
| Oat Flour | No aftertaste, dull up front stevia flavor, |
| | no new flavors introduced |
| Corn Flour | No aftertaste, mild up front stevia flavor, |
| | no new flavors introduced |

*All media made with 8 g/L of material, the corn/oat sample being made with 5 g/L and 3 g/L respectively. Product was tasted at 300 ppm 60% reb A and 0.8 ppm extracellular material powder.

### Example 4

The C. sinensis extracellular material powder (bitter blocker) is produced by the methods outlined in Example 4 and used with food products on a ppm basis.

TABLE 2
Bitter Blocker Concentration in Various Final
Bitter Blocking Product Applications*

| | Recommended Bitter |
|---|---|
| | Blocker Concentration (ppm) |
| Steviol Glycoside Mixture | 0.40-1.20 |
| Acesulfame - K | 0.3-1 |
| Aspartame | 0.3-1 |
| Chocolate | 35,000-37,000 |
| Tea | 1,066-1,866 |
| Red Ginseng | 180-220 |
| Zeviva Cola | 0.4-2.0 |
| Coffee Grinds | 7,800-73,000 |
| Coffee Brew | 100-500 |
| 100% Cranberry Juice | 50-3,200 |
| Coconut Water | 100-500 |
| Merlot | 600-3,800 |
| Tequila | 6,400-25,600 |
| Potassium Chloride | 40-60 |
| Vodka | 100-300 |
| Quinoa | 20-30 |
| Amaranth | 40-60 |

*Table 2 does not show how the bitter blocker is formulated into some of these products before application.

### Example 5

A 6:1 quinoa flour to basic bread flour was made where 25 ppm of the bitter blocker was added as a dry ingredient during kneading. The dough was baked in a Cuisinart CBK-100 series automatic bread-maker on the gluten free setting. A control dough without the bitter blocker was made under the same circumstances. It was concluded in multiple taste tests between 8 different people that the flavor of the treated bread was much less bitter and without the characteristic quinoa aftertaste. A similar experiment was conducted with a 1:1 amaranth flour to whole wheat flour mix where the bitter blocker was added at 50 ppm. The same results were observed by the same tasters.

### Example 6

A C. sinensis culture that had been cultured for 2.5 days at 25° C. in a bioreactor was vacuumed through a 25 μm filter. The filtrate was pasteurized, concentrated and spray dried. The resulting powder was added to a vitamins and mineral nutraceutical mix at 100 ppm. The resulting vitamin/mineral nutraceutical mix was noticeably less bitter and metallic to tasters. The powder derived from the culture filtrate was also used successfully to suppress the bitterness of OTC cough syrups when added up to 1,000 ppm.

Table 3 shows the amount of CLEARTASTE to use with dairy/dairy substitute to achieve a bitter blocking effect.

TABLE 3

| | Product | CLEARTASTE | CLEARTASTE |
|---|---|---|---|
| Product | concentration | ppm | % |
| Yogurt Greek 0% fat | | 20 | 0.002 |
| Non-dairy creamer, | 1.1-2.2% in | 2 | 0.0002 |
| powdered | brewed cinfee | | |
| Plant Based Greek | | 20 | 0.002 |
| Style Yogurt | | | |

Table 4 shows the amount of CLEARTASTE to use with nutritional products to achieve a bitter blocking effect.

TABLE 4

| | Product | CLEAR- | CLEAR- |
|---|---|---|---|
| | concen- | TASTE | TASTE |
| Product | tration | ppm | % |
| Protein Bars (General) | | 25 | 0.0025 |
| Chocolate Protein | | 35 | 0.0035 |
| Shake RTD | | | |
| Flavored Vitamin | | 5 | 0.0005 |
| Supplemented | | | |
| Beverage - No Calories | | | |
| Superfood Bars | | 20 | 0.002 |
| D-Calcium Pantothenate | 5% in | 50 | 0.005 |
| (B-Vit 5/Pantothenic | water | | |
| Acid) | | | |
| Probiotic Fruit Beverage | | 7 | 0.0007 |
| Vegan Protein Shake | 10% in | 50 | 0.005 |
| | water | | |

Table 5 shows the amount of CLEARTASTE to use with grains to achieve a bitter blocking effect.

TABLE 5

| | Product | CLEARTASTE | CLEARTASTE |
|---|---|---|---|
| Product | concentration | ppm | % |
| Millet flour, | 12.5% in batter | 75 | 0.0075 |
| in pancakes | | | |
| Sorghum flour, | 12.5% in batter | 35 | 0.0035 |
| in pancakes | | | |
| Sorghum flour, | 51% in dough | 25 | 0.0025 |
| in cookies | | | |

Table 6 shows the amount of CLEARTASTE to use with supplements and herbal ingredients to achieve a bitter blocking effect.

TABLE 6

| | | CLEAR- | CLEAR- |
|---|---|---|---|
| | Product | TASTE | TASTE |
| Product | concentration | ppm | % |
| Vitamin powder RTM | 5% in water | 9 | 0.0009 |
| RTM Branched Chain | 2% in water | 75 | 0.0075 |
| Amino Acid Blend | | | |
| Anhydrous Caffeine | 0.1% in water | 10 | 0.001 |
| Caffeine, in energy bar | 0.6% in bar | 15 | 0.0015 |
| Citrus Aurantium PE 30% | 0.08% in water | 30 | 0.003 |
| Quercetin Dihydrate 95% | 0.2% in water | 30 | 0.003 |
| Green Tea PE 30% | 0.08% in water | 15 | 0.0015 |
| Red Ginseng Concentrate | 3.1% in water | 500 | 0.05 |
| Baobab | 15% in water | 7 | 0.0007 |

Table 7 shows the amount of CLEARTASTE to use with protein ingredients to achieve a bitter blocking effect.

TABLE 7

| | | CLEAR- | CLEAR- |
|---|---|---|---|
| | Product | TASTE | TASTE |
| Product | concentration | ppm | % |
| Pea Protein | 7% in water | 20 | 0.002 |
| Isolate Organic (80%) | | | |
| in protein shake | | | |
| Potato Protein | 21% in water | 80 | 0.008 |
| Soy Protein | 3% in water | 50 | 0.005 |
| Rice Protein | 7% in water | 48 | 0.0048 |
| Brown Rice Protein, | 7% in water | 10 | 0.001 |
| Organic | | | |
| Whey Isolate + | 22% in water | 40 | 0.004 |
| Amino Acids | | | |
| Plant Protein | 13% in water | 8 | 0.0008 |
| Powder --Blend | | | |
| (Soy, Wheat, Pea) | | | |
| Fermented Soy Powder | 1.7% in water | 1 | 0.0001 |

Table 8 shows the amount of CLEARTASTE to use with miscellaneous ingredients to achieve a bitter blocking effect.

TABLE 8

| | Product | CLEARTASTE | CLEARTASTE |
|---|---|---|---|
| Product | concentration | ppm | % |
| Soluble Corn Fiber | 50% in water | 50 | 0.005 |
| Plant Based Greek Style | | 20 | 0.002 |
| Yogurt | | | |
| Potassium Chloride (KCl) | 2% in water | 40 | 0.004 |
| Cake, sugar free (stevia | | 27 | 0.0027 |
| sweetened) | | | |
| Dark Chocolate Cocoa | 1% in | 7 | 0.0007 |
| | Lowfat Milk | | |
| Baker's Chocolate | | 175 | 0.0175 |

### Example 7

Table 9 shows the amount of CLEARTASTE to use with sodium chloride and potassium chloride that was used to achieve a bitter blocking effect.

TABLE 9

| | Product | CLEARTASTE | CLEARTASTE |
|---|---|---|---|
| Product | concentration | ppm | % |
| Potassium Chloride (KCl) | 2% in water | 40 | 0.004 |
| NaCl/KCl (20% Na/ | 1% in water | 42 | 0.0042 |
| 80% K) | | | |
| Blend-for Table Salt | | | |
| replacement | | | |
| NaCl/KCl (30% NaCl/ | 1.5% in water | 2 | 0.0002 |
| 70% KCl) | | | |
| Blend-for Table Salt | | | |
| replacement | | | |
| Salt replacer (0% NaCl) | 2% in water | 40 | 0.004 |
| for Table Salt replacement | | | |

### Example 8

Mixtures of KCl and NaCl, with or without CLEARTASTE, are tested in a Temporal Check-All-That-Apply (TCATA) test. TCATA extends the Check-All-That-Apply (CATA) method. Selection and deselection of attributes are tracked continuously over time, permitting assessors to characterize the evolution of sensory changes in products. The total time interval is 60 seconds in each test. The task involves checking and unchecking words to track changes in the sample over time, such that at any given moment the words that are checked completely describes the sample at that moment.

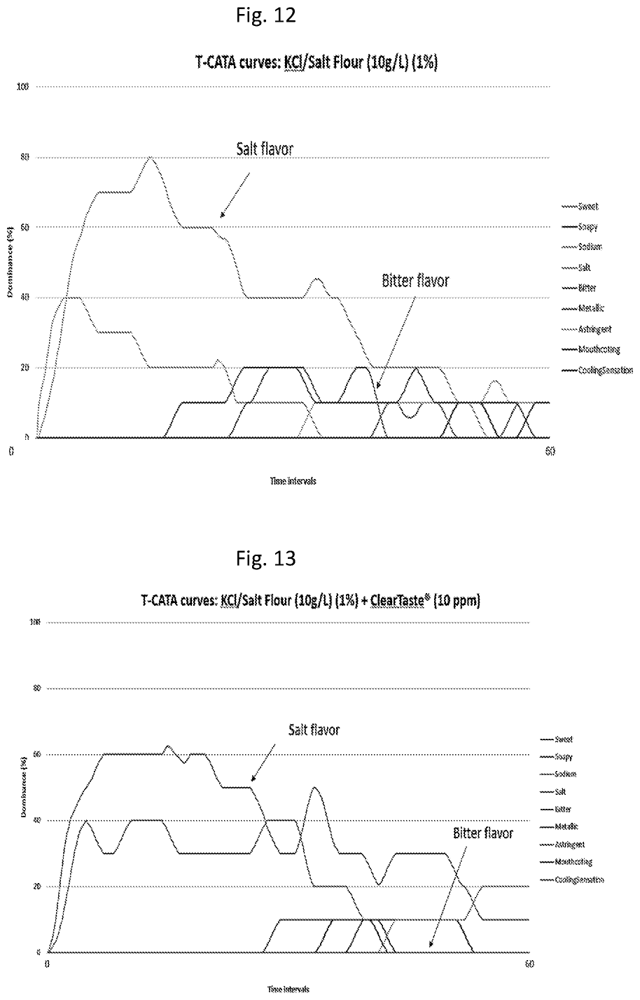

FIG. 12 shows a T-CATA curve for a KCl/NaCl flour (FLAKE SELECT KCl/salt fine flour, Cargill, Wayzata, Minn.; approximately 50% KCl and 50% NaCl by weight) at 1% compared with FIG. 13, T-CATA curve for a KCl/NaCl flour (FLAKE SELECT KCl/salt fine flour, Cargill, Wayzata, Minn.) at 1%, containing CLEARTASTE at 10 ppm, for ten individuals. The results showed that bitter flavor was noticeably reduced in the sample with CLEARTASTE. Specifically, bitter flavor in the control was shown to occur at levels of up to 20 dominance (%) from about 20 seconds to about 40 seconds, whereas the sample containing CLEARTASTE showed no bitter tastes. The results also showed that the salt flavor perception was extended in the sample containing CLEARTASTE. Specifically, at about 40 to 60 seconds, in the control, the salt perception was at 20 dominance (%) to 10 dominance (%) in the control, whereas in the sample with CLEARTASTE, in the time period between about 40 and about 60 seconds, the salt perception was at about 30 dominance (%).

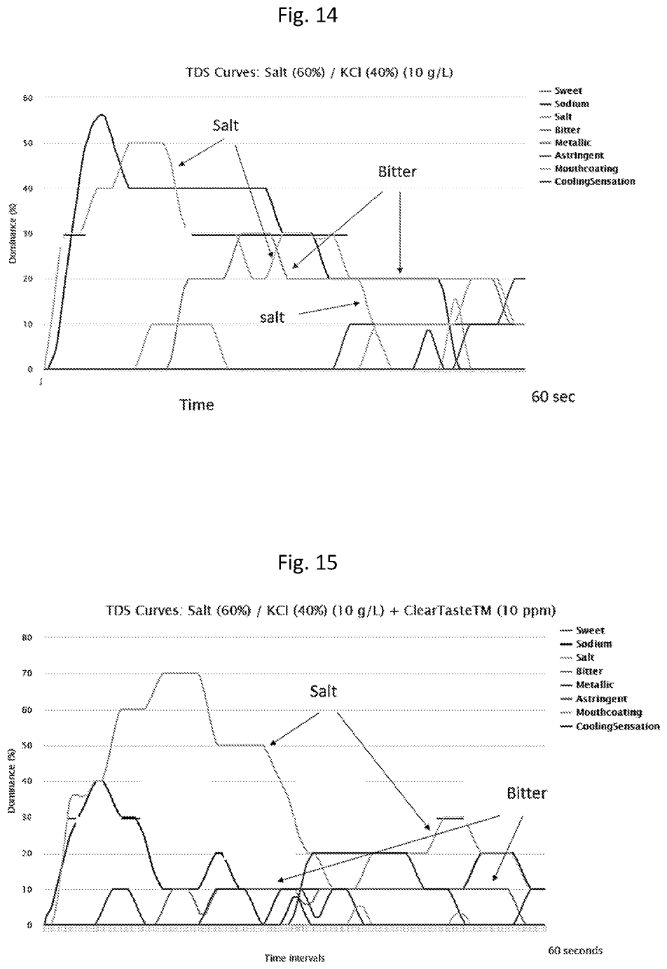

FIG. 14 shows TDS (similar to T-CATA) curves for a granular formulation of 60% NaCl and 40% KCl and FIG. 15 shows TDS curves for the a granular formulation of 60% NaCl and 40% KCl with 10 ppm CLEARTASTE. Specifically, a bitter taste is apparent in FIG. 14 at about 10 seconds and remains at approximately 20 to 30 dominance (%) throughout the tasting period. On the other hand, with CLEARTASTE, the bitter taste is at a lower level, 10 dominance (5) throughout the tasting period. The results also showed that the salt flavor perception was extended in the sample containing CLEARTASTE. Specifically, at about 45 to 60 seconds, in the control, the salt perception was at 0 dominance (%) with a very brief spike of −15 dominance (%) in the control, whereas in the sample with CLEARTASTE, in the time period between about 45 and about 60 seconds, the salt perception was at about 20 dominance (%) with a spike to 30 dominance (%). See FIG. 15. These results show that CLEARTASTE lowers the bitterness in combinations of KCl and NaCl and also extend the salty perception of flavor over the control.

### Example 9

Sensory Effects in Sprague-Dawley Rat Lick Studies of CLEARTASTE on Bitter and Sour Tastants and a High Intensity Sweetener

Bitter blocker prepared as described above in Example 2, at 5, 25, 50 and 100 mg/L alone (called CLEARTASTE herein) and added to each solution below:

Sucrose 250 mM; Saccharin 10 mM; MSG 150 mM; NaCl 400 and 800 mM; Citric Acid 10 mM; Quinine 0.08 and 0.16 mM.

16 naïve, adult (>50 days old, approximately 200 gram body weight), male Sprague-Dawley rats were tested in the MS-160 “Davis Rig,” manufactured by DiLog Instruments, Inc. We measured licking behavior at a resolution of 1 ms during the controlled presentation of up to 16 taste stimuli. The Davis Rig is housed inside an acoustic isolation chamber utilizing a white noise generator. Intake and exhaust fans located on opposing walls of the chamber direct constant air flow along the longitudinal axis of the stimulus delivery tray in order to reduce olfactory cues for any given stimulus. Therefore all licking behavior during brief (30 s duration) trials was motivated by orosensory/taste sensations. Rats were trained to lick during trials presenting water as the stimulus in the Davis Rig for three consecutive days prior to the start of testing. Each daily test session contained two blocks of 15 trials producing two replications of each test stimulus within each daily session. We tested the rats for a total of eight days.

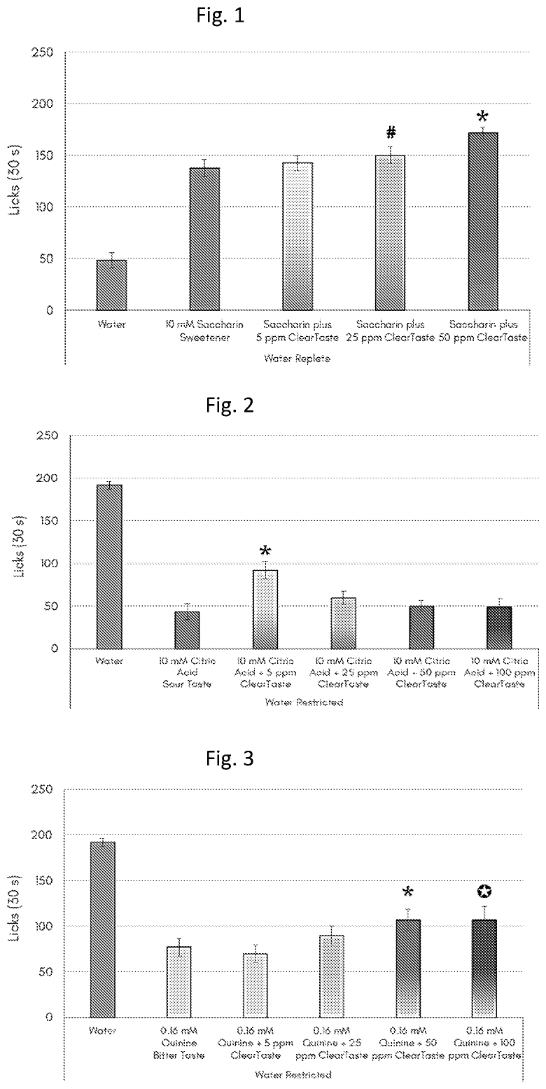

Saccharin. There was a significant increase in licking to 10 mM saccharin and all saccharin plus CLEARTASTE solutions compared to licks to water under water replete (not thirsty) motivation. There was a significant main effect of CLEARTASTE concentration [F(3, 45)=12.847, p<0.001] with post-hoc paired t-tests revealing a significant increase in licking for saccharin plus 25 ppm CLEARTASTE compared to saccharin alone [t(15)=2.163, p=0.047] and a significant increase licking for 50 ppm CLEARTASTE added to saccharin compared to all other stimuli [saccharin alone t(15)=4.618, p<0.001; 5 ppm CLEARTASTE+saccharin t(15)=5.853, p<0.001; 25 ppm CLEARTASTE+saccharin t(15)=3.862, p=0.002]. See FIG. 1, showing appetitive rat-lick study of CLEARTASTE in saccharin at various concentrations.

Citric acid. There was a significant main effect of CLEARTASTE concentration [F(4, 60)=6.602, p<0.001] with post-hoc paired t-tests revealing a significant increase in licking compared to 10 mM citric acid alone when 5 ppm CLEARTASTE [t(15)=3.606, p=0.003] was added to the citric acid solution. The effect of CLEARTASTE at 5 ppm and lack of effect at subsequent higher concentrations has been mirrored in previous sensory tests conducted by a yogurt producer on sourness and dairy notes in their own yogurt. See FIG. 2, aversive rat lick study of CLEARTASTE in citric acid.

Quinine. There was a significant main effect of CLEARTASTE® concentration [F(4, 60)=5.349, p=0.001] with post-hoc paired t-tests revealing a significant increase in licking compared to 0.16 mM quinine alone when 50 ppm CLEARTASTE® [t(15)=3.418, p=0.004] and 100 ppm CLEARTASTE® [t(15)=2.018, p=0.062] were added to 0.016 mM quinine. See FIG. 3, aversive rat lick study of CLEARTASTE in quinine at 0.16 mM. 0.16 mM quinine (70 licks) was significantly more aversive than 0.08 mM quinine (170 licks) compared to water (192 licks). This increase in averseness revealed a significant effect of CLEARTASTE at ≥50 ppm to reduce the aversive bitter taste component. 0.016 mM quinine (70 licks) was significantly more aversive than 0.08 mM quinine (170 licks) compared to water (192 licks). This increase in averseness revealed a significant effect of CLEARTASTE at ≥50 ppm to reduce the aversive bitter taste component. Given that the increase in licking (decrease in averseness) was moderate (approximately 30 additional licks) and was similar for 50 and 100 ppm, it might be interesting to test higher CLEARTASTE concentrations to see if that is the ceiling of the bitter-blocking effect or if we can further suppress the bitter aversive taste properties by increasing CLEARTASTE concentrations.

### Example 10

SELECTIVE EFFECTS OF BITTER BLOCKER (called CLEARTASTE® herein) on bitter taste receptors.

Six bitter taste compounds selective for TAS2R receptors and two broadly stimulating compounds in combination with CLEARTASTE® were tested to identify selective effects of CLEARTASTE® on bitter taste receptors. CLEARTASTE was added at 25, 50 and 100 ppm (dry weight) to four concentrations of each of the below taste solution representing six selective activations of specific bitter taste receptors, one broad activation of bitter taste receptors and ethanol alcohol. Amygdalin selective activates TAS2R-16; erythromycin selectively activates TAS2R-10; ethylpyrazine selectively activates TAS2R-38, noscapine selectively activates TAS2R-14; acetaminophen selectively activates TAS2R-39; hydrocortisone selectively activates TAS2R-46; diphenidol broadly activates TAS2R-1, -4, -16, -38, -39, -47, and -49; and ethanol was tested at 5, 7, 10, and 12% v/w.

16 naïve, adult (>50 days old, approximately 200 gram body weight) male Sprague-Dawley rats were tested in the MS-160 “Davis Rig.” Procedures were as described for Example 9.

Licking responses of 16 rats to 4 concentrations of 8 bitter taste compounds plus water with and without the addition of 25, 50 and 100 ppm CLEARTASTE were measured. This resulted in testing 128 taste compounds with and without CLEARTASTE. Also measured were 32 tests of water (n=8) and CLEARTASTE dissolved in water in the absence of tastants (25, 50 and 100 ppm n=8). This provided a robust replication of previous findings that CLEARTASTE 25-100 ppm dissolved in water is not aversive compared to water alone in motivated (thirsty) rats. The testing identified two TAS2R bitter taste receptors (R-16 and R-46) that were blocked by CLEARTASTE and two TAS2R bitter taste receptors (R-10 and R-14) that were unaffected by CLEARTASTE. CLEARTASTE added to diphenidol (which stimulates multiple bitter taste receptors including R-38 and R-39) exhibited a larger masking capacity of bitter taste receptors than a potentiation effect. The action in diphenidol was likely due to the ability of CLEARTASTE to reduce bitterness in most product applications, since most products likely act on multiple TAS2R receptor and not primarily through just the TAS2R-38 and -39 variants.

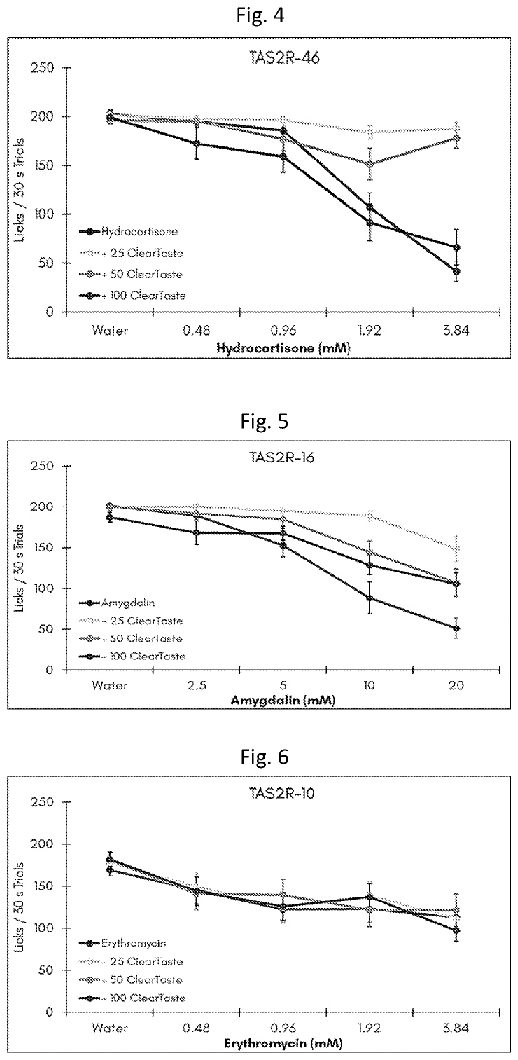

There were differential antagonistic effects for increasing concentrations of CLEARTASTE. The optimal concentration to block both the TAS2R-16 and -40 receptors was 25 ppm. CLEARTASTE was effective on R-46 at both 25 and 50 ppm using hydrocortisone. See FIG. 4. CLEARTASTE was effective at blocking the R-16 bitter taste receptor at all concentrations using amygdalin. See FIG. 5. CLEARTASTE at 25 ppm completely eliminated the bitterness of the two highest concentrations of hydrocortisone (R-16), see FIG. 5, and produced 100% elimination of the bitterness of the next to highest concentration of amygdalin (R-46)(FIG. 5) with a 288% increase in licking to the highest amygdaline concentration representing only a 25% decrease in licking compared to water (see FIG. 5.).

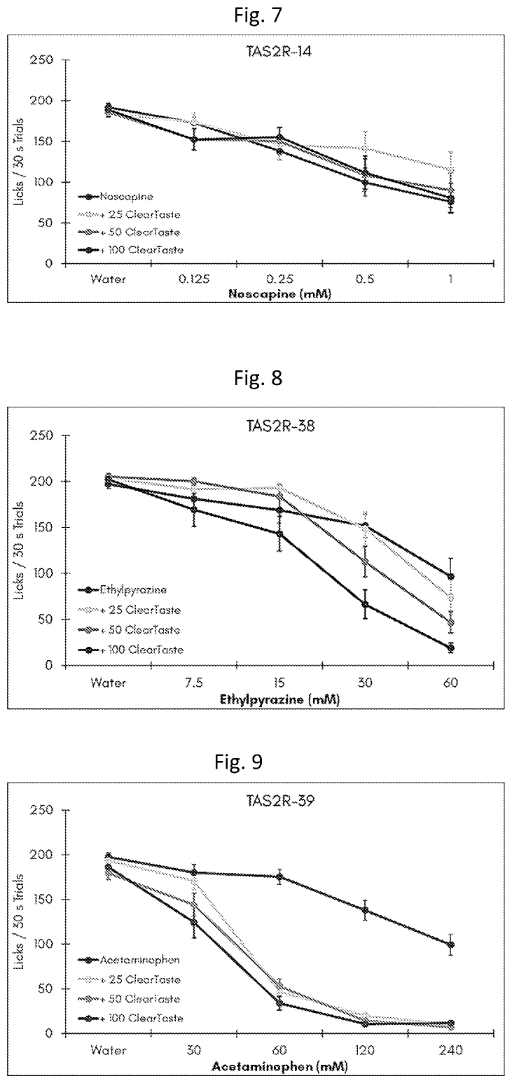

Summary results: at 25 ppm, CLEARTASTE effectively blocks bitterness due to TAS2R-46 (hydrocortisone), CLEARTASTE effectively blocks bitterness due to TAS2R-16 (amygdalin), CLEARTASTE has less effect on bitterness due to TAS2R-10 (erythromycin) (FIG. 6). CLEARTASTE has less effect on bitterness due to TAS2R-14 (noscapine) (FIG. 7). CLEARTASTE has less effect on TAS2R-38 (ethylpyrazine) (FIG. 8.)

There was a main effect for the increasing concentration of ethylpyrazine to reduce licking [F (4, 60)=89.466, p<0.001] as an indication of increasing bitterness of the taste chemical. There was a significant main effect of CLEARTASTE concentration [F (3, 45)=13.129, p<0.001] indicating that as increasing concentrations of CLEARTASTE were added to ethylpyrazine licking decreased indicating that the bitterness increased. There was a significant interaction between the ethylpyrazine concentration and CLEARTASTE [F (12, 180)=3.107, p=0.001] indicating that the 50 & 100 ppm CLEARTASTE enhanced the bitter taste signal of concentrations ≥30 mM while the 25 ppm CLEARTASTE was not different from ethylpyrazine alone.

Ethylpyrazine selectively activates the TAS2R-38 bitter taste receptor. CLEARTASTE at 25 ppm appears to have a lesser effect on the TAS2R-38 receptor. Additionally, CLEARTASTE is not effective at blocking TAS2R-39 activation (acetaminophen) at certain concentrations (FIG. 9).

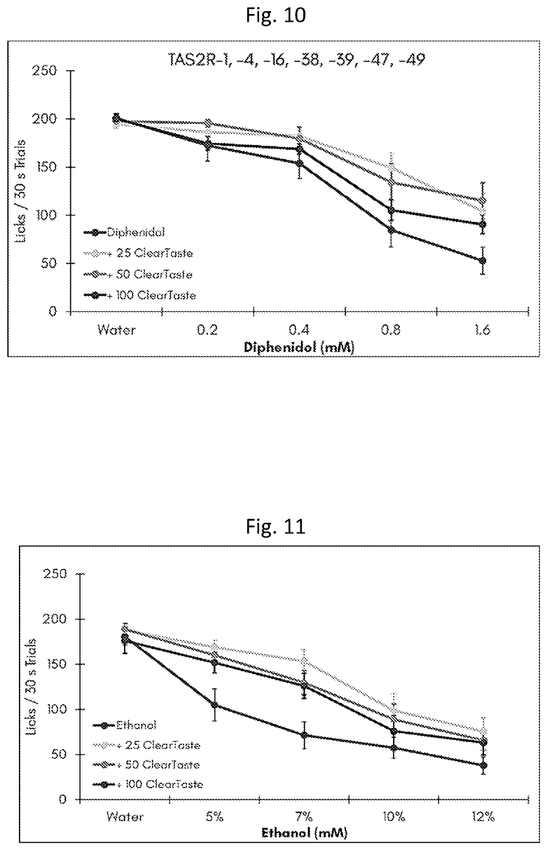

CLEARTASTE reduces bitter taste due to diphenidol (TAS2R-1, -4, -16, -38, -39, -47, -48) (see FIG. 10). There was a main effect for the increasing concentration of diphenidol to reduce licking [F (4, 60)=39.754, p<0.001] as an indication of increasing bitterness. There was a significant main effect of CLEARTASTE concentration [F (3, 45)=10.157, p<0.001] indicating that 25 & 50 ppm CLEARTASTE reduced the bitterness more than 100 ppm CLEARTASTE. There was a significant interaction between the diphenidol concentration and CLEARTASTE [F (12, 180)=2.258, p=0.011] indicating that the 25 & 50 ppm CLEARTASTE only had an effect on reducing bitterness once diphenidol was sufficiently bitter at concentrations ≥0.8 mM.

Diphenidol broadly activates at least seven different bitter taste receptors. Most bitter taste compounds would also activate multiple different taste receptors, so this represents a general ability of CLEARTASTE to reduce bitterness by acting through multiple different bitter taste receptors.

CLEARTASTE reduces averseness to ethanol at 5, 7, 10, and 12% v/w. See FIG. 11. There was a main effect for the increasing concentration of ethanol to reduce licking [F (4, 60)=64.157, p<0.001] as an indication of increasing bitterness. There was a significant main effect of CLEARTASTE concentration [F (3, 45)=5.689, p<0.001] indicating that CLEARTASTE reduced the averseness of ethanol particularly at the 5 & 7% ethanol concentrations. There was a significant interaction between the ethanol concentration and CLEARTASTE [F (12, 180)=1.984, p=0.028] indicating that CLEARTASTE only had a significant reduction in averseness for 5 & 7% ethanol.

Ethanol is a multisensory stimulus eliciting both bitter (taste TAS2R-38 and TAS2R-3/4/5 variants) and astringency (tactile) sensations resulting in avoidance by rats as an aversive stimulus. All concentrations of CLEARTASTE effectively reduce the averseness of ethanol at concentrations <10% alcohol.

CLEARTASTE can be an effective enhancer of low alcohol content but appears less effective at alcohol doses greater than 10%.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a combination of at least one flavor compound and a taste-modulating portion of a filamentous fungus mycelial aqueous culture, wherein the mycelia is from a Cordyceps spp.

2. The composition of claim 1, wherein the at least one flavor compound is a combination of sodium chloride and potassium chloride.

3. The composition of claim 2, wherein the composition, when combined with a product for oral administration, reduces metallic and/or bitter taste resulting from the presence of KCl and provides an extended “salt” flavor perception (linger) in the product for oral administration during the first sixty seconds of tasting by T-CATA test, as compared to a combination that does not contain a taste-modifying portion from a mycelial aqueous culture.

4. The composition of claim 2, wherein the ratio of sodium chloride to potassium chloride in the flavor compound combination is approximately 50:50.

5. The composition of claim 1, wherein the at least one flavor compound is a bitterness blocker or a bitterness masker, wherein the combination is capable of synergistically reducing bitter tastes or reducing aftertastes of a product for oral administration compared to a control without the at least one flavor compound.

6. The composition of claim 5, wherein the at least one flavor compound is capable of blocking or masking bitter perception due to activation of the human bitter taste receptors TAS2R-38, TAS2R-39, TAS2R-10, and/or TAS2R-14 receptors by a product for oral administration.

7. The composition of claim 6, wherein human bitter taste receptors are TAS2R-14 and/or TAS2R-38.

8. The composition of claim 7 wherein the at least one flavor compound is selected from the group consisting of 6-methoxy flavanone or probenecid.

9. The composition of claim 1, wherein the taste-modulating portion from a mycelial aqueous culture is a mycelia-free taste-modulating portion from the mycelial aqueous culture or an extract of the mycelial liquid culture.

10. The composition of claim 1, wherein the fungus is selected from the group consisting of Cordyceps scarabaeicola, Cordyceps takaomontana, Ophiocordyceps dipterigena, Ophiocordyceps amazonica, Cordyceps cylindrica, Cordyceps sphecocephala, Metacordyceps martialis, Ophiocordyceps melonlonthae, Ophiocordyceps nutans, Ophiocordyceps curculionium, Ophiocordyceps australis, Ophiocordyceps Cordyceps caloceroides, and Cordyceps variabilis Cordyceps mililaris and Cordyceps sinensis.

11. The composition of claim 10, wherein the fungus is Cordyceps sinensis.

12. The composition of claim 11, wherein the taste-modulating portion is a dried taste-modulating portion.

13. The composition of claim 1, wherein the taste-modulating portion of the mycelial aqueous culture is prepared by a method comprising:
(i) culturing a mycelial aqueous submerged culture in a media;
(ii) separating the extracellular material fluid from the mycelial cells; and
(iii) collecting the extracellular material fluid of the mycelial liquid culture as the mycelia-free taste-modulating portion;
or
culturing a mycelial aqueous culture in a media;
(ii) heating the mycelial aqueous culture to between about 60° C. and 100° C. for between about 10 minutes and 80 minutes;
(iii) separating the extracellular material fluid from the mycelial cells; and
(iv) collecting the extracellular material fluid of the mycelial liquid culture as the extract taste-modulating portion,
wherein the mycelia comprises a mycelia from a Cordyceps spp.

14. The composition of claim 13, further comprising pasteurization or sterilization of the extract taste-modulating portion or mycelia-free taste-modulating portion of the mycelial aqueous culture.

15. The composition of claim 3, wherein the product for oral administration is a dietary supplement, a food product, a food additive, a pharmaceutical, or a nutraceutical.

16. A method for enhancing the taste of a product for oral administration, comprising:
(A), comprising
(i) culturing a mycelial aqueous culture in a media;
(ii) separating the extracellular material fluid from the mycelial cells; and
(iii) collecting the extracellular material fluid of the mycelial liquid culture as the mycelia-free taste-modulating portion;
(B) adding at least one flavor compound to the collected mycelia-free taste-modulating portion or collected extract taste-modulating portion to form a combination; and
(C) adding the combination to the product for oral administration in an amount sufficient to enhance the product's taste.

17. The method of claim 16, wherein the at least one flavor compound is a combination of sodium chloride and potassium chloride.

18. The method of claim 17, wherein the flavor enhancement comprises reduced metallic and/or bitter taste resulting from the presence of KCl and an extended “salt” flavor perception in the product for oral administration during the first sixty seconds of tasting by T-CATA test, as compared to a combination that does not contain a taste-modifying portion from a mycelial aqueous culture.

19. The method of claim 17, wherein the ratio of sodium chloride to potassium chloride in the flavor compound combination is approximately 50:50.

20. The method of claim 16, wherein the at least one flavor compound is a bitterness blocker or a bitterness masker, wherein the combination is capable of synergistically reducing bitter tastes or reducing aftertastes of a product for oral administration compared to a control without the at least one flavor compound.

21. The method of claim 20, wherein the at least one flavor compound is capable of blocking or masking bitter perception due to activation of the human bitter taste receptors TAS2R-38, TAS2R-39, TAS2R-10, and/or TAS2R-14 receptors by a product for oral administration.

22. The method of claim 16, wherein the taste-modulating portion from a mycelial aqueous culture is a mycelia-free taste-modulating portion from the mycelial aqueous culture or an extract of the mycelial liquid culture.

23. The method of claim 18, wherein the fungus is Cordyceps sinensis.

24. A method to improve a product for oral composition's taste, comprising administering with the product a composition according to claim 1.
